(19) United States
(12) Patent Application Publication
(10) Pub. No.: US 2026/0026755 A1
(43) Pub. Date: Jan. 29, 2026

(54) Signature Extraction Using Self-Supervised Motif Discovery

(71) Applicants: Somayeh Bakhtiari Ramezani, Starkville, MS (US); Amin Amirlatifi, Starkville, MS (US); Shahram Rahimi, Starkville, MS (US); Maria Seale, Starkville, MS (US)

(72) Inventors: Somayeh Bakhtiari Ramezani, Starkville, MS (US); Amin Amirlatifi, Starkville, MS (US); Shahram Rahimi, Starkville, MS (US); Maria Seale, Starkville, MS (US)

(21) Appl. No.: 19/279,130

(22) Filed: Jul. 24, 2025

Related U.S. Application Data
(60) Provisional application No. 63/675,008, filed on Jul. 24, 2024.

(51) Int. Cl. A61B 5/00; A61B 5/318

(52) U.S. Cl. CPC A61B 5/7267; A61B 5/318

(57) ABSTRACT

A self-supervised technique capable of identifying an expected number of motifs in a given sequential or temporal set and extracting them. A robust algorithm that is used to identify unique motifs and their signature, coupled with a proper distance metric to compare the signatures of partially similar motifs.

### CROSS REFERENCES TO RELATED APPLICATION

PRIORITY OF U.S. PROVISIONAL PATENT APPLICATION Ser. No. 63/675,008, FILED Jul. 24, 2024, INCORPORATED HEREIN BY REFERENCE, IS HEREBY CLAIMED.

### STATEMENTS AS TO THE RIGHTS TO THE INVENTION MADE UNDER FEDERALLY SPONSORED RESEARCH AND DEVELOPMENT

NONE

### BACKGROUND OF THE INVENTION

### Field of the Invention

The present invention pertains to a self-supervised technique capable of identifying an expected number of motifs in a given sequential or temporal set and extracting them. More particularly, the present invention pertains to a robust algorithm that is used to identify unique motifs and their signature, coupled with a proper distance metric to compare the signatures of partially similar motifs.

### Brief Description of the Prior Art

The advancements in acquiring real-time data generated by various sources, such as sensors, wearable devices, and medical records, have given rise to the detection of meaningful patterns in large temporal data. Extracting these patterns enables us to gain valuable insight into the system's state and predict its future. Analyzing these large bodies of temporal data, however, requires specialized algorithms to extract meaningful patterns from them.

An electrocardiogram (ECG or EKG) signal is a sequence of time series data representing electrical impulses from the heart muscle and recorded from multiple electrodes placed on the skin. It is a preferred noninvasive tool for cardiac disease detection by physicians, characterized by its multiple signal recordings and periodic waveform synchronized with the cardiac cycle. Detecting anomalies in ECGs is crucial for identifying cardiac issues. However, existing anomaly detection methods often suffer from a high rate of false alarms due to the difficulty of distinguishing ECG artifacts from genuine signals, which in turn poses challenges for accurate diagnosis and interpretation, thus necessitating improved techniques in this field.

Several of the techniques commonly used for temporal data analysis consume the data directly and without considering its global structure. Examples of such approaches include using statistical modeling (autoregressive integrated moving average, exponential smoothing, seasonal decomposition), Machine Learning (ML) algorithms such as Recurrent Neural Networks (RNNs), data clustering, classification algorithms, and visualization techniques (e.g., using charts, scatter plots, and heat maps). Given that in complex datasets, each activity session can consist of a long sequence of patterns that form different global structures, applying ML algorithms on such data without first identifying the local patterns is not feasible and would result in low performance.

One way of exploring temporal data is to extract the sequence of patterns in them, commonly referred to as the motifs. A prominent example of Motifs can be seen in the pattern of general operations (such as walking, running, washing hands, or sleeping) captured through a wearable sensor (such as a smartwatch). However, motifs can also be observed in repetitive operations like driving straight or taking a turn, financial market patterns, and healthy or unhealthy trends (such as the normal operation of a manufacturing robot vs. failure of one of its bearings).

When examining system states to detect abnormal or faulty patterns, it is possible to encounter more than one pattern and situations where the state is unknown. This is due to the fact that prior knowledge of the types of motifs that need to be sought after is not necessarily known. As a result, one needs to be able to discover motifs in an unsupervised manner. Motif extraction methods can be generally classified as supervised and unsupervised. In supervised motif extraction, a sequence of temporal data is defined as the target, and different segments of the data are examined to identify possible matches. Unlike supervised methods, where a specific motif is sought throughout the data, an unsupervised extraction aims to extract all motifs, both repeated and rare ones.

From the theoretical analysis perspective, supervised motif discovery algorithms are regarded as search problems, whereas unsupervised motif extraction counts as an enumeration problem, i.e., the hardest type of problem. While supervised motif discovery is already well explored, the same argument cannot be made for unsupervised analysis of temporal data, which requires algorithms that can extract all possible motifs. Although semi-supervised motif extraction is also entertained, most of the current work on semi-supervised algorithms for time series data focuses on classification and clustering.

The extraction of unique local patterns and establishing a database of prototypes or signatures is an important first step in analyzing long temporal data, such as ECGs. To achieve this, three main tasks are defined for temporal data analysis, namely the extraction of motifs, their comparison and identification, and automatic label generation for similar motifs.

One of the challenges faced by motif discovery algorithms is the correct determination of the window size, a critical task in many different applications. In ECGs, as an example, since each person has different cardiac cycles, the beat lengths can change depending on their respiratory drives. In other words, the beat window can vary from one patient to the next under different activities that a person conducts or even how they breathe. To overcome this limitation and extract motifs from ECGs, prior work used the motif discovery algorithms introduced by others, with respect to a minimum description length.

Another challenge faced by motif discovery algorithms is to identify whether a motif is frequent or rare. To compare an extracted motif against other parts of data, an adequate similarity metric needs to be used that is capable of handling noise in the data and calculating the distance between patterns with different lengths.

### SUMMARY OF THE INVENTION

The present invention pertains to a robust framework for self-supervised motif extraction to identify and categorize unique motifs and their signature. The present invention also introduces a unique distance metric to compare the signatures of partially similar motifs, assigning a degree of semblance between the two. Moreover, the present invention brings the following non-exhaustive contributions to the field: (1) Introducing a technique for identifying the number of patterns in a long temporal set without the need for prior labeled data or human intervention; (2) A new distance metric for motifs' signature comparison that can handle different levels of noise, thus providing valuable insights into the degree of resemblance between motifs; (3) Introduction of Self-supervised motif discovery with the goal of extracting all meaningful motifs and categorizing them together; and (4) Advancing knowledge in motif analysis by adding explainability to highlight the differences between motif signatures.

The present invention pertains to a self-supervised technique capable of identifying the expected number of motifs in a given sequential or temporal set and extracting them. More particularly, the present invention pertains to a robust algorithm that is used to identify unique motifs and their signature, coupled with a proper distance metric to compare the signatures of partially similar motifs. Defining such distance metrics allows for assigning a degree of semblance between two motifs that may have different lengths or contain noise. In addition, the present invention further adds explainability to the proposed approach by showing the differences between any two motifs.

The framework is composed of two main modules: the Motif Extraction (ME) module, as well as the Pattern Identification and Explanation (PIE) module.

The ME module uses spectrogram and peak finding algorithms to detect and extract motifs. These motif signatures are then extracted and compared with one another to identify unique prototypes. Each unique prototype is stored, forming a repository of observed motifs and allowing for the assessment of similarity between newly discovered motifs and those previously observed. The PIE module uses a decision-based algorithm to enhance an understanding of different prototypes and their defining features that set them apart.

### BRIEF DESCRIPTION OF THE DRAWINGS/FIGURES

The foregoing summary, as well as any detailed description of the preferred embodiments, is better understood when read in conjunction with the drawings and figures contained herein. For the purpose of illustrating the invention, the drawings and figures show certain preferred embodiments. It is understood, however, that the invention is not limited to the specific methods and devices disclosed in such drawings or figures.

FIG. 1 depicts the architecture of the framework used in the present work. Raw temporal data are subjected to the Motif Extraction (ME) module, where motifs are extracted and tested against previously observed motifs. Unique motifs are persisted and can optionally be labeled by an expert for semi-supervised studies. The Pattern Identification and Explanation (PIE) module employs DT to discern distinctive features that distinguish various motifs from one another.

FIG. 2 depicts results of applying different time-frequency domain transformations to a snippet of ECG recordings. It can be noted that the ECG contains two abnormal recordings at locations 275 and 1600. These abnormalities are reflected as spectral changes in transformed data shown in the STFT, CWT, and WVT.

FIG. 3 depicts a three-dimensional (3D) spectrogram of ECG signal in FIG. 2 shows the variation of amplitudes over time. This plot can be used to determine the frequency ranges to break the data and find a minimum threshold that can be used to split the data.

FIG. 4 depicts index mapping between windowed sections of the spectral data, and the original dataset.

FIG. 5 depicts a connectivity mask that is used to determine the neighboring points or pixels of each spectrogram entry. This square matrix assigns a value of 1 or True to connected points while assigning a value of 0 to all other neighbors. A 4-Connectivity (left) and 8-Connectivity (right) masks were used. It is noted that the 8-Connectivity mask outperforms the 4-Connectivity mask, delivering three times better results without compromising accuracy or performance.

FIG. 6 depicts an example of raw ECG data and spectral conversion. The peaks detected in the spectral data are displayed as red dots.

FIG. 7 depicts a Symbolic Fourier Approximation binning approach.

FIG. 8 depicts a proposed approach for comparing two signatures for partial similarity. Signature s₁ of length h is considered partially similar to signature s₂ of length l₂, when the absolute value of subtracting their array of bin numbers results in an array of zeros in d positions and non-zero elements less than a predefined permissible degree of difference, A, in permitted positions.

FIG. 9 depicts the UCR ECG example in its raw form.

FIG. 10 depicts the results of global structure detection and peak finding modules on a snippet of a simple periodic set.

FIG. 11 depicts self-supervised behavior, where the motif with signature acedb and a length of 1,950 samples has formed a cluster on its own, while the other two motifs both share the same signature (aadecb), but their length difference has set them apart.

FIG. 12 depicts a CPU run-time demand of the motif extraction algorithm versus the number of data points in a simple periodic set.

FIG. 13 depicts the memory usage of the motif extraction algorithm for the same set of experiments.

FIG. 14 depicts that the GPU is about 34.6% faster than the CPU in processing larger size sample sets (e.g., for 1.00e+7 and 1.00e+8 samples).

FIG. 15 depicts a complex synthetic set example presented in the top image and is comprised of two sets with repetitive patterns. In addition, the motif extraction module has successfully identified the beginning and end points of each pattern, as depicted by the dots in the bottom image, which correspond to the location of peak spectral changes, indicated by the bars in the bottom image.

FIG. 16 depicts the results of clustering of the motifs.

FIG. 17(a) depicts two sets of normal pattern prototypes identified in the UCR ECG data.

FIG. 17(b) depicts the PVC signals that are captured and clustered together. It is important to note one major difference in a T-wave portion (or the tail part) of Pattern 5 versus Pattern 9 that has likely resulted in their separate clustering.

FIG. 18 depicts an anomalous signal captured by the framework of the present invention as Pattern 7.

FIG. 19 depicts a special case in the analysis of the present invention, where unusual patterns are singled out.

FIG. 20 depicts an overall look at the frequency bins that are formed by each pattern from MIT-BIH Sample 102.

FIG. 21 depicts the top three most important features that are used in identifying different prototypes.

FIG. 22 depicts a breakdown of the number of unique prototypes versus their respective Leaf ID in the decision tree.

FIG. 23 depicts a clear explanation of the decision rules that govern the decision tree created for the MIT-BIH sample 102.

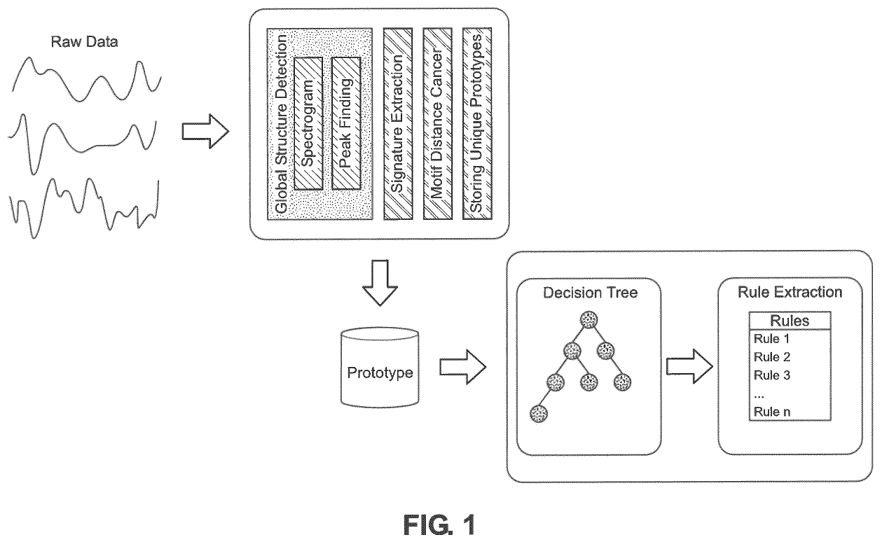

### DETAILED DESCRIPTION OF A PREFERRED EMBODIMENT

### Motif Extraction

Having an efficient technique for extracting motifs is a critical component of the proposed framework. Most of the existing motif extraction algorithms are supervised methods based on fixed motif length, averaging techniques, and analyzing the two together. Although this technique is prevalent and has been used in various applications, it has a drawback that prevents it from fully capturing motifs that begin in one window and extend over multiple windows. To address this drawback, the present invention uses a set of components in the ME module that allow for variable pattern length.

### 1.1 Using Spectral Data to Detect the Global Structure

Signal analysis can be conducted using their spectrum, which represents the distribution of their energy or power across different frequencies. This type of analysis gives insights into the statistical properties and average behavior of the signal at different frequencies. One of the most convenient ways of visualizing the spectrum of the frequency content of a signal over time is to use a spectrogram. In this form of visualization, a plot of time versus the signal frequency is constructed, with the color or intensity showing the strength or magnitude of the captured frequency content at each point.

The Fourier Transform (FT) is a valuable tool for examining stationary and periodic signals and reveals information about their frequency components; however, it falls short when it comes to showing the time of specific frequencies. This constraint becomes even more apparent when working with non-stationary signals that have changing frequency content over time.

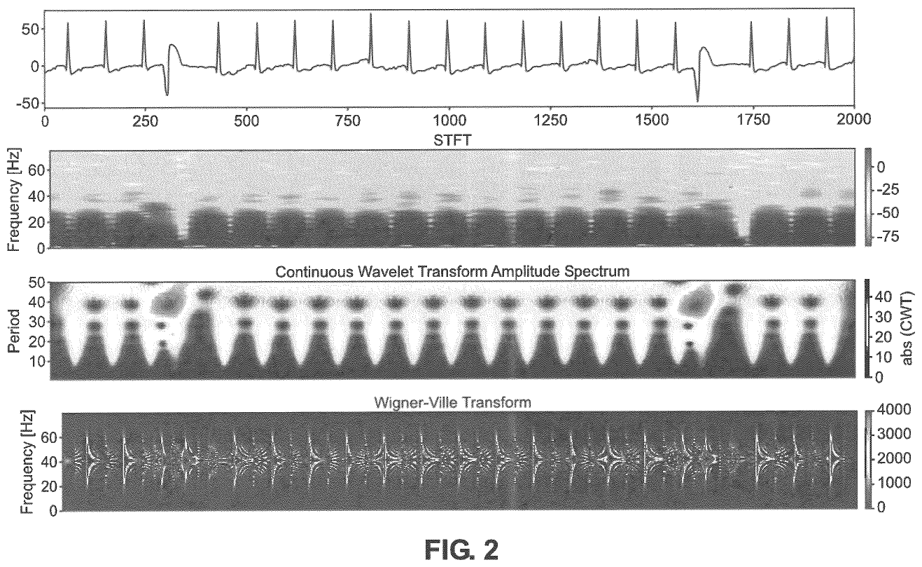

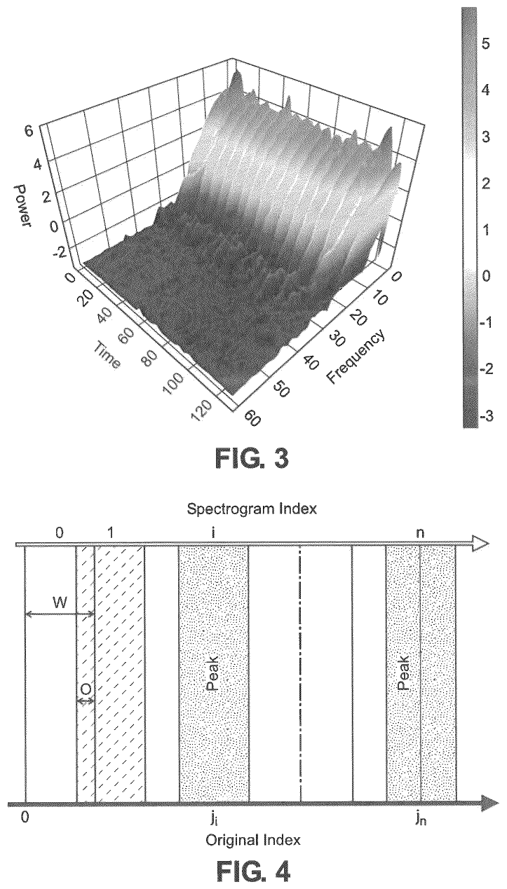

The main techniques to convert a signal to the time-frequency domain, as depicted in FIG. 2, include defining a moving window and applying the Short-Time FT (STFT), Continuous Wavelet Transform (CWT), or the Wigner-Ville Transform (WVT). These approaches produce a dynamic representation of the data instead of the FT, which offers a snapshot of the frequency components. This invention uses the STFT for its subsequent analysis due to its simplicity and versatility.

Changes in the power spectrum can be used to identify transitions in the state of a system. Spectral changes in the data can give insight into its global structure. The present invention uses time STFT to determine whether significant changes in the power spectrum of temporal data can effectively discern the underlying global structure of the data and accurately identify segmentation points.

In order to make this determination, the optimal spectral model setup with adequate resolution and accuracy needs to be established. The spectral resolution and temporal accuracy are highly dependent on window length. Similar to the Heisenberg uncertainty principle, which is a well-known phenomenon in quantum mechanics and molecular dynamics, focusing on the time aspect of a wave would result in the loss of frequency accuracy, and aiming for precise calculation of frequency would result in a reduction of temporal resolution. In other words, the length of a window in time-frequency analysis is a trade-off between frequency precision and temporal precision. Although narrow windows offer excellent temporal accuracy, there need to be more data points in them to calculate frequencies precisely. Longer windows have more data points, allowing for more precise frequency information, but because the temporal activity is integrated across a longer time frame, they also have lesser temporal precision. Additionally, it needs to be considered that using a small window would result in a small number of sampled points and excessive calculations.

The present invention uses the Gabor transform in the form of a spectrogram function to detect the global structure of data. It breaks the input data into smaller chunks before performing an FFT on each of them. The length of each chunk, NFFT, is defined as a hyperparameter.

A degree of overlap between neighboring windows can be defined between neighboring windows so that no cross-window event is gone unnoticed.

The output of the spectrogram function is a 2D spectrum, with each column reflecting the periodogram of succeeding windows. This output is used as a fundamental input to the “peak-finding” module. The function also returns the time values associated with the windows' midpoints (i.e., the rows of the “spectrum” array), which are used in “index-mapping” function to reflect the index of peak-bearing windows back to their position in the original data.

### 1.2 Peak Finding

A crucial part of the present invention as a framework is to perform a peak-finding step. A peak is defined as the local maximum of a signal; thus, detecting changes in the slope of a signal can indicate a peak. Peaks often indicate important events and finding them can help identify the characteristics of the signal. There are many different ways to find peaks in a signal, with the simplest approach involving finding all local maxima by a simple comparison of neighboring values. This can be achieved with simple presumptions that a peak always has a higher value than its surrounding points. Another basic approach to finding peaks in signals is based on the observation that a peak has a value higher than a certain threshold.

Using a CWT based on the first derivative of a Gaussian function is another effective way of peak finding, as it is sensitive to the changes in the slope of the signal. This technique is called the Ridger wavelet peak detection algorithm, as the CWT results in ridges when dealing with low signal-to-noise ratio (SNR) and significant shifting baseline component; however, most of the existing peak finding algorithms fail when it comes to detecting peaks in noisy periodic and quasi-periodic data.

Spectral peaks can tolerate noise and possible significant signal impairments. Furthermore, the peak magnitudes can be disregarded and only point pairs indicating the time and frequency of the peaks would be persisted. In doing so, this can transform an otherwise complicated spectrogram into a few point pairs.

Peak finding in spectrograms involves identifying local maxima that correspond to amplitudes of the frequency component in the original signal; thus, techniques that were discussed earlier (such as the local maxima search and the ridge detection) are also applicable here; however, as has been established, the spectrogram represents the frequency content of a signal over time, meaning that peak finding in a spectrogram involves detecting high-intensity areas in a two-dimensional array, which requires traversing in both vertical and horizontal directions. One needs to use a sliding window to examine for areas with high intensity or large values to find peaks in a spectrogram. The size of this window needs to be set slightly larger than the expected width of the peak to be detected to ensure that the peak is fully captured. It is worth noting that the peak finding window is not related to the STFT window and can be different from that.

Once the appropriate window size is established, the algorithm can search for the largest value within each window and examine it against a set of criteria set forth by the user. If the selected value meets these criteria, such as the value being above a certain threshold or significantly higher than the neighboring points, that point is subsequently identified as a peak.

Peak-finding is implemented in relatively small regions to identify time-frequency points with a magnitude greater than their neighbors within a specific neighborhood, as expanding the size of the region would make it more challenging to select a point. Once a peak is identified, it is used as the representative value of the window to which it belonged. As illustrated in FIG. 4, a mapping needs to be established between windowed sections of the spectral data and the original dataset. This index mapping needs to account for the size of the windows, w, used in calculating the spectral data, and any overlaps, o, that were considered between neighboring windows. The following equation shows the mapping that takes place:

$${j}_{t} = {( i + 1 )}^{*} w / 2 + {i}^{*} {o}^{*} w$$

Here jₜ is the index of the peak in the raw data space, i is the index of the window in spectral space, w is the window length used to create the spectral data, and o is the overlap between each window that is used to create the spectral data.

Implementation. Peaks are used as the segmentation points to break the original raw data into motifs or patterns. In order to identify these peaks, the present work has liberally adapted and expanded the implementation. This is achieved by constructing the spectral data and defining the global structure, then determining the local maxima of each window in the resulting 2D array. The present work defines several hyperparameters tailored toward specific frequency ranges or amplitude thresholds to help calibrate the model for each dataset.

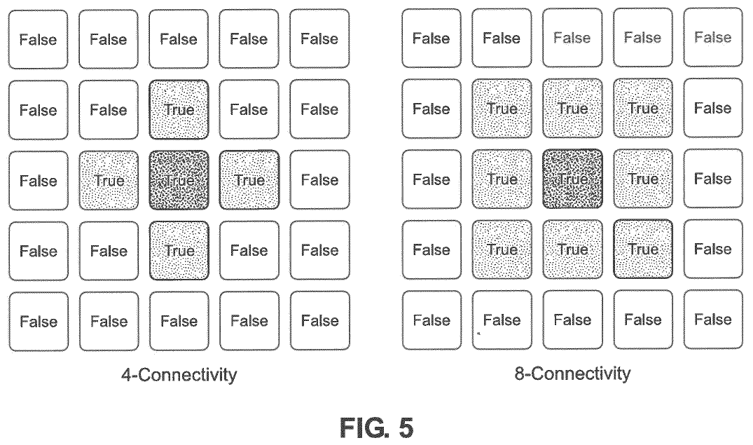

Detection and construction of a list of tuples representing peaks, consisting of their frequency and time, depends on the user-defined minimum amplitude threshold. Each entry is then treated in the 2D spectral set as a pixel in the spectrogram image. This allows for the generation of a connectivity mask to define the neighborhood of each spectrogram entry or pixel. This mask forms a square matrix, where connected points are assigned a value of 1 or True, while all other neighbors are assigned a value of 0. In the present work, two connectivity masks are utilized, as depicted in FIG. 5. It is worth noting that the 8-Connectivity mask outperforms the 4-Connectivity mask, exhibiting three times better results without compromising accuracy or performance.

A two-pronged approach is taken to reduce noise and unwanted artifacts in the data. First, a neighborhood mask is used to ensure that peaks are surrounded by neighbors that also have high amplitudes. Next, a binary background operation is used to remove insignificant peaks.

Using the neighborhood mask helps us reduce the number of false positives in peak detection. In this context, false positives refer to peaks that result from noise, rather than being the actual peaks. The neighborhood mask can be used to identify these false positives by expanding the boundaries of the mask to include neighboring pixels. This is achieved by generating a binary structure out of the mask and then dilating it. The Dilation is a morphological operation used to expand the boundaries of the neighborhood mask by iterating over the structure several times and adding the neighboring pixels to the mask. This results in a mask that is larger than the original structure and includes all of the pixels that were originally in the structure, as well as their neighbors. If a peak is surrounded by other pixels that are themselves high in amplitude, then it is more likely to be an actual peak rather than a false reading or noise. By doing this, we can reduce the number of false positives and improve the accuracy of our peak detection algorithm.

To further reduce noise in the spectrogram, a binary background erosion operation is performed on the spectral data using the neighborhood mask that was generated earlier. This will help remove any remaining noise not captured by the neighborhood mask. To this end, all elements of the original spectral data with a zero value are designated as the background (binary value equal to 1), and the rest of the elements are taken as the foreground. Any peaks previously identified through local maxima search are tested against the resulting eroded background, and those that coincide with the background are discarded, as they are likely false positives. This will help further to improve the accuracy of our peak detection algorithm.

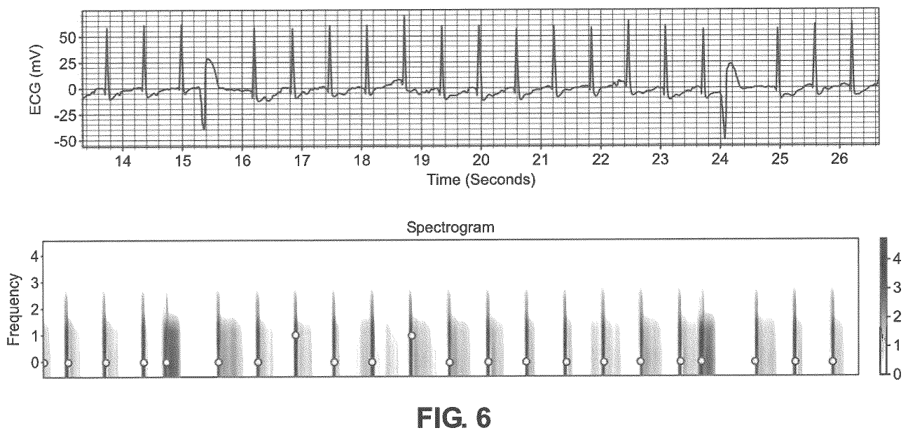

In the present research, the spectral amplitudes are allowed to be real-valued on a linear scale and accommodate for absolute values on a logarithmic scale, as different applications (e.g., ECGs vs. audio signals) may require different resolutions. FIG. 6 shows an example of ECG data together with its spectrogram and peak detection results.

### 1.3 Signature Extraction

The Symbolic Fourier Approximation (SFA) technique is adapted to extract words of length=with letters ranging from 0-through an alphabet size of Nₐ from each identified motif. This technique was originally presented as a way of extracting symbols from short-time series data sets in order to classify them.

Definition 1.1: The signature of motif mi is defined as s₁ with a length of l₁, which corresponds to the first l₁ Fourier Coefficients (FCs) of m. Considering c€ N as an offset, then c≤FCs≤(c+h).

Definition 1.2: Given an alphabet of Σ={a, . . . , z}, where |Σ|=Nₐ, an amplitude bin is defined as the equal representation of the observed range of amplitudes, divided by the size of the alphabet, Nₐ.

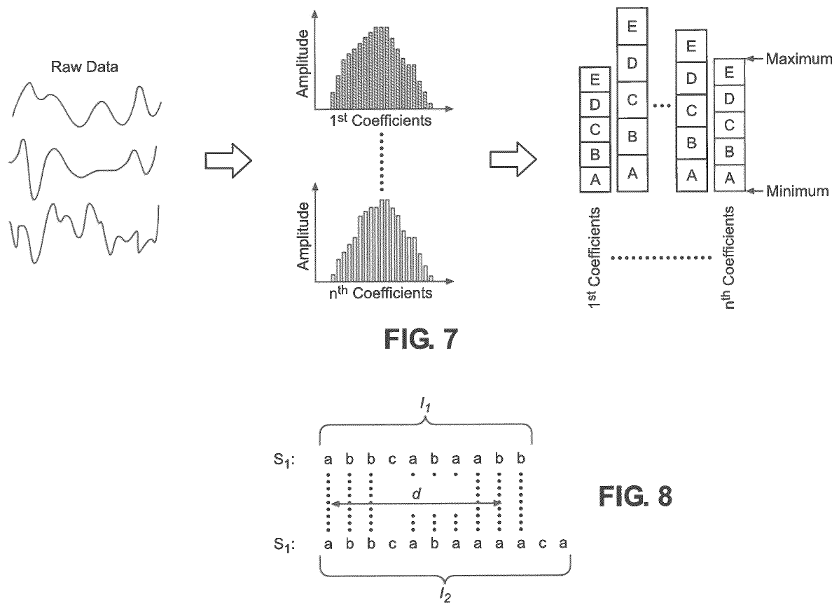

As illustrated in FIG. 7, an FT is applied on each extracted motif to identify its FCs. To convert the FCs into their symbolic representations (i.e., words), each FC amplitudes range is broken down into a set of Nₐ bins, where Nₐ is the size of the alphabet used to form the word. In the present work, these words are referred to as signatures.

Unlike the original work, where amplitudes and phases are used to form bins, the present work only uses amplitudes to perform binning and create signatures of motifs. Additionally, an equal frequency division is used for binning (FIG. 7), where the observed frequency range is divided into Nₐ bins. Another possible binning approach is to use an equal number of frequency populations in each bin; however, the current framework seldom uses this approach.

The process of extracting signatures consists of two steps: (1) Learning phase; and (2) Execution phase.

During the learning phase, a subset of the data is taken as the training set. The percentage of the data used for this task is anywhere from 20% to 80% of the data, depending on the desired task. The training data is used to determine the frequency ranges that make up each bin.

Once the bins are set up, in the execution phase, the rest of the data is subjected to FC extraction. Depending on the resulting FCs, each observation coefficient is assigned to one of the bins that were set up during the learning phase. This results in an array of size Nₐ of bin numbers, which is used as the motif signature. Conversely, this array of bin numbers can be transformed into alphabetic equivalents for easier interpretation by the user. As an example, an observation of [1, 0, 2, 0, 3], whose alphabet Σ={a, b, c, d}, translates into bacad, i.e., a word with the length of five. In other words, we are reducing a motif, which could be composed of several hundred data points, into a five-letter long signature comprised of the first five FCs of that motif.

### 1.3.1 Implementation

The signature extraction module is a critical component of the approach to motif clustering. By extracting informative and comparable signatures, the accuracy of our clustering algorithms can be improved. The signature extraction module is adapted from the original work and expanded to meet the requirements and desired outcome of the present invention. One such expansion is using a normalization step to ensure that the signatures are comparable across different datasets.

To extract the signatures, the extracted motifs are split into training and testing sets. The percentage of data used for training and testing can vary depending on the specific application. However, the present invention uses 20% of the data for training the signature extraction module and the rest for testing/execution.

However, some applications considered in the present work used a larger training set (e.g., 80% of the data) to create a model that can distinguish anomalous data. One such example was ECG anomaly detection, where the goal was to classify as many motifs as normal, marginal, or abnormal. This required sampling a larger part of the data to ensure that the majority (if not all) of the possible frequency ranges are observed to form the bins and subsequently form our signatures.

The alphabet size and signature length are set by the user. As noted earlier, the equi-frequency approach is used to divide the range of minimum to maximum frequencies' amplitudes into the desired alphabet size before the signature of each motif is calculated. Additionally, based on the task or dataset, we may use an offset for the minimum frequency by setting the normMean attribute to True. For example, we set the offset to 2 in ECG and synthetic data.

### 1.4 Motif Similarity Metric

As mentioned before, the majority of point-to-point comparison algorithms face limitations. To overcome such limitations, in the present work, a novel and efficient similarity metric for comparing different motif signatures has been introduced. While such comparisons can be made via string searches or regular expressions, the present invention uses the array of bin numbers for each motif signature. A simple element-wise subtraction of two motif signatures can show the degree of similarity or dissimilarity between the two (FIG. 8). Furthermore, the absolute values of the resulting differences can be used to enhance similarity assessments.

Definition 1.3: Signature s₁ of length l₁ is considered similar to signature s₂ of length l₂, when l₁=l₂ and subtracting their array of bin numbers results in an array of zeros.

Example: Signature s₁:[1, 0, 2, 0, 3] is similar to signature s₂:[1, 0, 2, 0, 3], as the element-wise difference of their bin number arrays results in [0, 0, 0, 0, 0].

Definition 1.4: Signature s₁ of length l₁ is considered partially similar to signature s₂ of length l₂, when the absolute value of subtracting their array of bin numbers results in an array of zeros in 3 positions, and non-zero elements less than a predefined permissible degree of difference, Δ, in permitted positions. Permitted positions denote the number of elements that can vary from each end of the array.

Example: If the permissible degree of difference is set to Δ≤2, then signature s₁:[1, 0, 2, 0, 2] is considered partially similar to signature s₂:[1, 0, 2, 0, 3], with a degree of difference of one from the right side, as the element-wise difference of their bin number arrays results in [0, 0, 0, 0, 1].

Implementation. In order to identify similar motifs and categorize the like ones, one needs to compare their signatures. As previously described in Section 1.4, the present invention performs an elementwise subtraction of the numeric values of signature elements to determine the degree of similarity between any two motifs. Two motifs are considered similar if their lengths are the same, and their difference results in an array of zeros. While this is a stringent form of similarity check, it is one that guarantees a perfect match between two motifs.

It should be noted, however, that an equal length of signatures does not necessarily imply, or require, the two motifs to have the same lengths. In fact, one of the strengths of the present research is the variability of the length of motifs, while their overall behavior is the same. To explain this point further, consider a normal heartbeat pulse under different activity levels. When a person is resting, the number of beats per minute (BPM) is generally 60-100 BPM, which is less than that of active times, 120-150 BPM. This suggests that the length of each beat signal would be shortened during active times, compared to those observed during resting times; however, both of these beats can be considered normal beats. On the contrary, a premature ventricular contraction (PVC) can exhibit a completely different signature; thus, there should be no similarity between a normal heartbeat and a PVC. This is exemplified in FIG. 6, with normal heartbeats showing an upward peak, whereas the two PVCs observed at 15.5 seconds and 24.1 seconds exhibit a downward trend.

### 1.5 Storing Unique Motifs

Once all motifs and their corresponding signatures are extracted, the similarity check is performed on the resulting motifs, and a set of unique motifs, along with their partially similar subsets, are created. This set of unique motif signatures is kept as reference signatures. While the proposed framework is considered to be unsupervised, a domain expert can be tasked with labeling the observed unique signatures for future comparisons and anomaly detection. Additionally, any existing labels in the data (e.g., labels for ECG data) can be used at this stage to denote the master behavior of each unique motif signature.

### 2. Prototype Identification and Explanation

The set of unique motif signatures kept as the reference signatures are referred to as the prototypes. These prototypes are used for subsequent analysis and comparisons, where each new signature is tested against the prototypes' signatures.

Definition 2.1: A Prototype P is a motif calculated as an average value of all similar motifs clustered together through their similarity metric.

The present invention strives to explain the underlying conditions that result in healthy and faulty behaviors of a system. Different levels of explainability exist, but the most understandable form is achieved through decision rules. A decision rule shows the path taken through a tree and the split decisions made at nodes. One can readily extract decision rules from tree-based algorithms by combining split decisions taken at a node. This approach can be used against any tree-based algorithm, including ensemble tree algorithms, such as a random forest.

To work towards this goal, a decision tree is used to extract rules pertaining to each motif. This will allow us to determine the critical frequencies and amplitudes that differentiate between unique prototypes of motifs. This explainable AI (XAI) approach is achieved by feeding the sequence of signatures for each motif as the inputs to a tree-based algorithm. Next, the decision rules used by the tree will be examined. These rules can be readily transformed into conversational texts that explain the decision-making and splitting criteria used by the tree. This ability to extract textual rules from the identified signatures puts us in the white box region of the explainability spectrum, which is particularly useful in identifying the underlying states that cause a faulty motif vs. a healthy one.

While in many fields such as bioinformatics or DNA sequencing, determining the repeated patterns or frequent motifs is the goal, in other fields such as healthcare, the frequent motifs (e.g., normal heartbeats in a healthy patient) are considered trivial, expected, or less important. In such cases, a motif that is considered significant (e.g., an abnormal heartbeat pattern) can be one that is rare and not frequent. Ranking of motifs would allow for the quantification of their uniqueness and their degree of importance.

Definition 2.2: For a range R, we can define the Most Significant Motif (MSM) in the time series T as the sub-sequence of length n, which contains the highest number of non-trivial matches. This sub-sequence is referred to as the S¹, shown in the following equation: S¹=1−Motif (n, R) where n is the length of the subsequence, R is the range to be searched to find the motif, and 1 is the degree of motif significance.

The definition of MSM can change depending on the use case. For example, in RUL prediction, MSM is defined as:

Definition 2.3: The MSM for RUL is defined as the motif that is observed in the majority of samples, has a significant number of occurrences, and shows a higher frequency of occurrence towards the end of the life of training samples.

Implementation. In order to extract the rules and create an XAI approach, the present invention has used the Fast Interpretable Greedy-tree Sums (FIGS) module of the iModel library. FIGS is based on the Classification and Regression Trees (CART) model, which is often used for classification and regression. It grows an ensemble of decision trees, rather than using a single tree, in a greedy form which makes the model more interpretable. This makes it easier to understand how each tree contributes to the overall prediction. It considers all trees that have already been grown and selects one that would most reduce the total unexplained variance in the data and grows a new tree from its residuals.

The present invention uses the dtreeviz to visualize the decision trees and validate the extracted rules generated by FIGS Classifier. dtreeviz is a Python library that can visualize decision trees. It can display the extracted decision rules in a tree-like structure, along with the feature importances, thereby allowing for the ability to visually inspect the decision rules and how they are applied to the data and also to be able to identify any potential problems. One such example was to look for rules that are over-complex or those that do not seem to be supported by the data.

### 3. Results and Discussion

The following section herein discusses the results of applying the motif discovery technique of the present invention to four distinct datasets: two sets of synthesized data and two sets of ECG data. The two ECG data sets offer a collection of electrocardiogram recordings, which give us an opportunity to evaluate the applicability of our proposed motif discovery algorithm in detecting abnormal cardiac patterns. The ability to detect recurring motifs in ECG data is instrumental in the early diagnosis of cardiac abnormalities and in improving patient outcomes.

As mentioned previously, the synthetic data sets are specifically designed to assess the capabilities of the proposed motif discovery algorithm. By employing a variety of techniques, such as random noise generation, seasonality, and pattern manipulation, the synthesized data provides a controlled environment for evaluating the accuracy and efficiency of motif discovery.

The results obtained from this dataset contribute valuable insights into the performance and limitations of the technique of the present invention under controlled conditions. Additionally, the simple periodic set allowed for testing the scalability of the algorithm on systems with limited resources such as the edge computers.

### 3.1 Simple Periodic Set

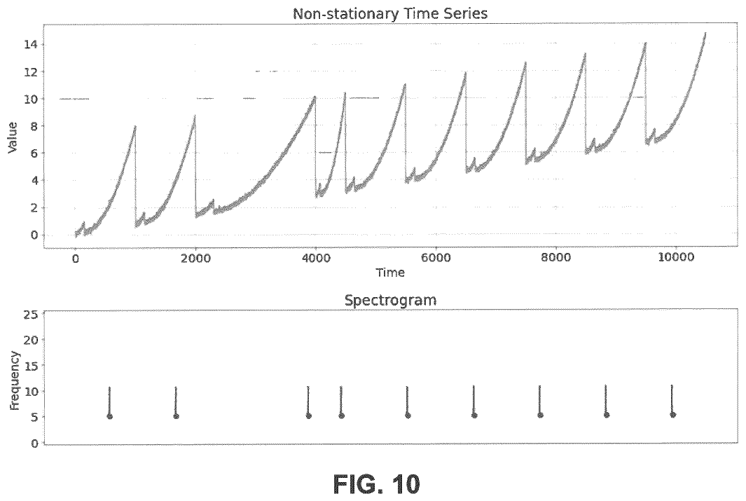

FIG. 10 depicts the results of global structure detection and peak finding modules on a snippet of the simple periodic set.

As depicted by the raw data (top part of the figure), this example is composed of ten patterns of exponential noisy data, each bearing a seasonal sub-pattern indicated by a small hump. In addition to the seasonality, the example also indicates an increasing trend, as shown by the gradual increase in the overall values. While both of these features are fairly simple to capture by most temporal data analysis packages, one thing that makes this set stand out is the unusual length of the third and fourth patterns.

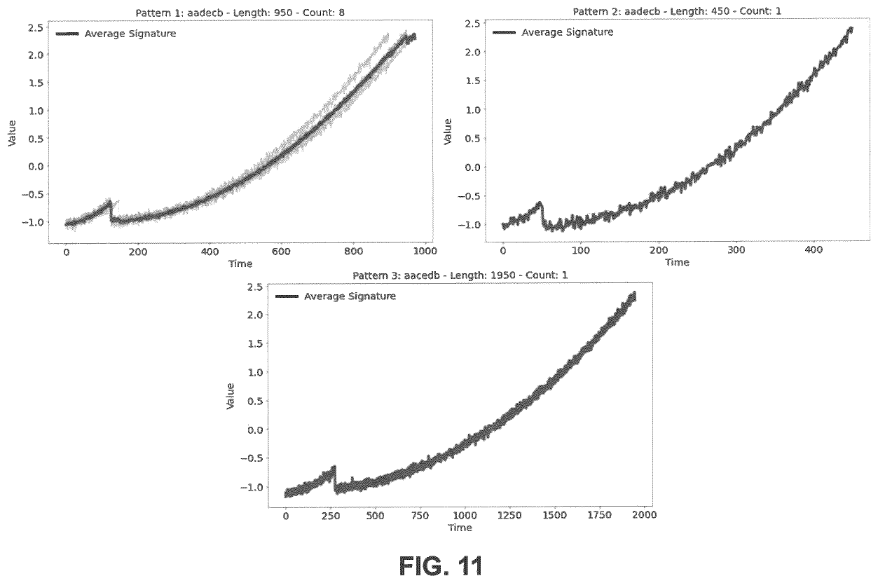

While the majority of patterns have a length of between 900 to 974 samples, the length of the third pattern has intentionally been increased to be 1,950 samples long and reduced the length of the fourth pattern to be 450 samples long. This variability in length can pose a challenge for most algorithms, but the framework of the present invention was able to detect these motifs, as depicted in FIG. 11.

As noted in FIG. 10, the motif extraction algorithm identified a total of nine break points, as indicated by the plurality of dots on the bottom plot. These breakpoints correspond to the location of peak spectral changes, as indicated by the plurality of bars in the same plot.

Despite the increasing trend that is indicated in this example, an observer setting the maximum degree of length difference to a small value (e.g., 10%) would expect to see three clusters of motifs in this example. In other words, if all motifs were Z-transformed, then we should expect to see the extra-long and extra-short motifs in two separate clusters, but the rest of the motifs that have an average length of 950 should be clustered together. This similarity is accurately captured by a signature extraction and a similarity check component. This self-supervised behavior is depicted in FIG. 11, where the motif with signature acedb and a length of 1,950 samples has formed a cluster on its own, while the other two motifs both share the same signature (aadecb), but their length difference has set them apart. This has resulted in eight of the motifs which share similar lengths (between 900 and 974) being clustered together, while the extra short motif, which shares the same signature, is clustered separately.

As a result, the framework of the present invention has successfully clustered the like motifs together while observing their length. It is to be noted that if the degree of length difference was set to a higher value (e.g., 60% or greater), then the extra short motif would have also been clustered with the rest of the medium-length motifs. This indicates that the framework of the present invention is capable of separating short motifs, or keeping them with the others, depending on a user's preference.

### 3.2 Scalable Periodic Set.

In an effort to examine the scalability of the framework of the present invention, a repetitive periodic dataset is created based on the simple periodic set, where the number of data points and patterns hidden in the data can be easily prescribed by the user. Similarly, memory usage is gathered by examining the state of memory_info of the process before and after the execution of each main calculation.

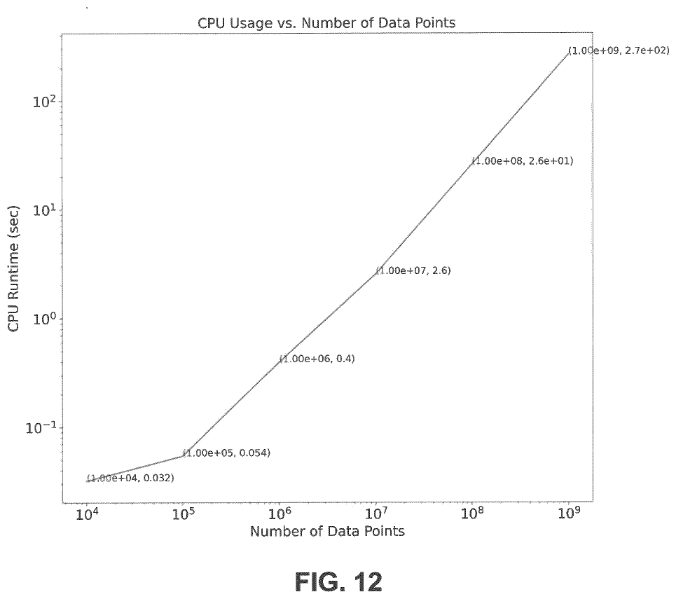

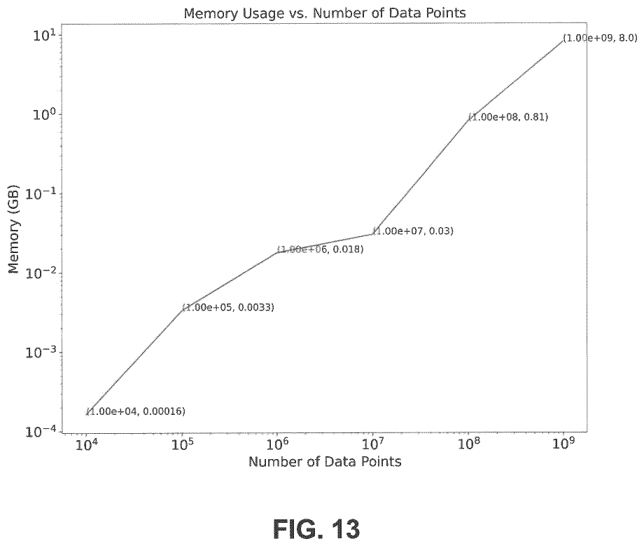

The benchmarks are performed on a single thread CPU of the workstation. FIG. 12 shows the CPU run-time demand of the motif extraction algorithm versus the number of data points in the simple periodic set. As shown herein, the CPU run-time increases at the same rate as the number of data points. Since the present invention is increasing the number of data points by an order of magnitude, the same exponential response is also observed in CPU runtime. Additionally, FIG. 13 depicts the memory usage of the motif extraction algorithm for the same set of experiments. A similar exponential trend is also observed for memory usage, with the exception of the 1.00e+7 set. These observations confirm that the algorithm of the present invention is capable of scaling with the data and can utilize high-performance computing resources if they are available.

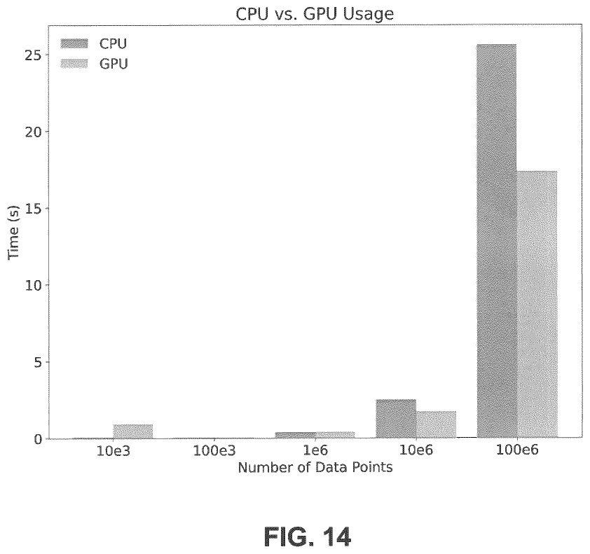

Next, the same benchmarks on the GPU are performed and compared to the results against the CPU. As depicted in FIG. 14, the GPU is about 34.6% faster than the CPU in processing larger size sample sets (e.g., for 1.00e+7 and 1.00e+8 samples). However, the GPU fails to exhibit similar behavior for smaller sample sizes (e.g., 1.00e+6 or less). This is a well-studied phenomenon when it comes to CPU versus GPU performance comparison. Among many factors that can affect the performance of a GPU, the size of the data set, the type of operations, and the specific algorithm being used, stand out. If the sample size is too small, as was demonstrated by the 1.00e+6 or less examples, the transfer of data between different components, including RAM, CPU, and GPU, would slow down the process more than it would see any speed up from performing the calculations in GPU.

### 3.3 Complex Synthetic Set.

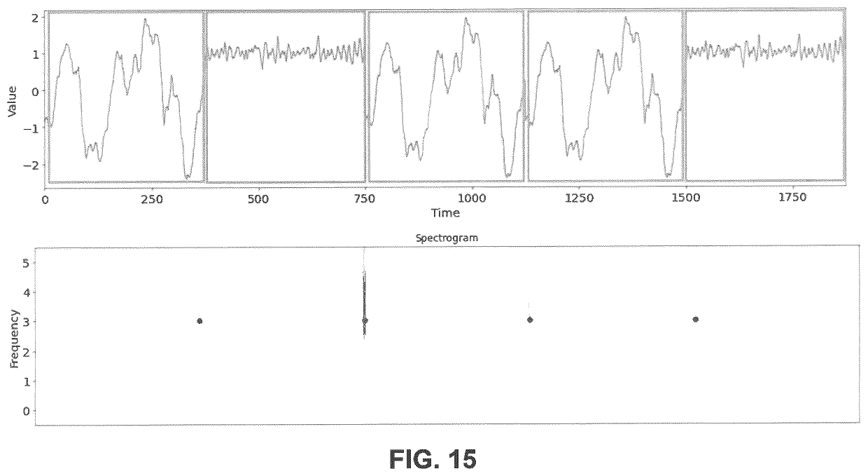

The complex synthetic set establishes a controlled environment to evaluate the robustness, precision, and effectiveness of the motif identification and extraction of the presentation invention by including a diverse set of obstacles, such as random noise insertion and pattern editing. The performance and constraints of this framework under controlled circumstances can be better understood by analyzing the findings from this dataset. The complex synthetic set example presented in the top part of FIG. 15 is comprised of two sets with repetitive patterns. The first, third, and fourth patterns are GP, while the second and fifth patterns are AR. As noted in FIG. 15, the motif extraction module has successfully identified the beginning and end points of each pattern, as depicted by the plurality of dots in the bottom plot. These points correspond to the location of peak spectral changes, indicated by the plurality of bars in the bottom plot.

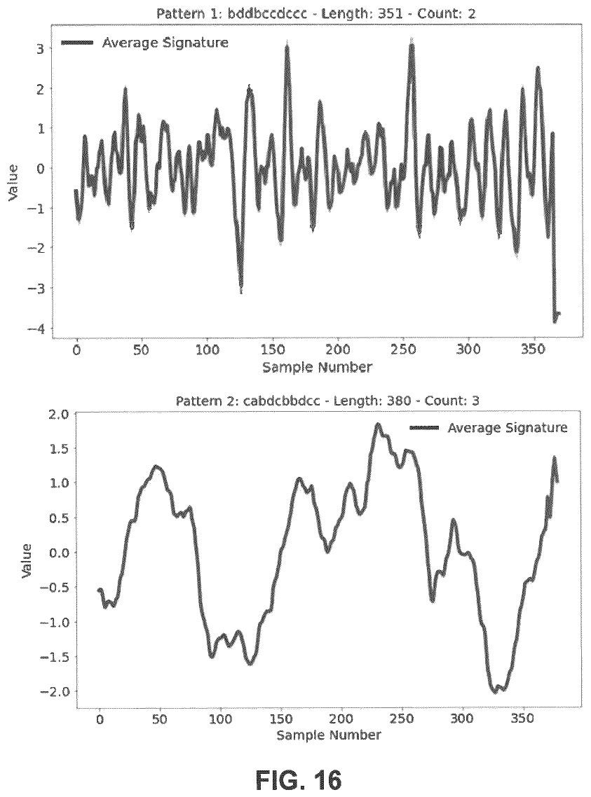

FIG. 16 depicts the results of clustering of the motifs. As expected, the clustering has resulted in three GP patterns overlaid on each other and two AR patterns clustered with each other separately. This observation confirms that the proposed framework is capable of capturing more complex motifs, even if the said motifs contain substantial degrees of noise and are surrounded by other motifs that exhibit different (as in the cases of AR vs. GP patterns) or similar (as is the case for the two successive GP motifs) patterns.

### 3.4 UCR Anomaly Benchmark Dataset

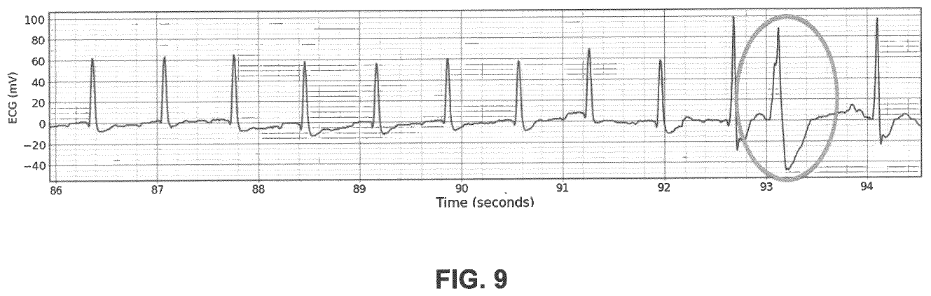

As described hereinabove, the UCR Anomaly benchmark dataset contains several different datasets, a few of which are based on ECG data. One such ECG dataset will be studied herein, which is especially challenging for anomaly detection. Under normal conditions, the PVCs are considered to be anomalous readings in an ECG, and many researchers have tried to identify them with advanced ML algorithms. What makes this dataset challenging, however, is that the PVCs are considered normal occurrences; instead, an artificial abnormality is inserted at one point in the data. FIG. 9 depicts the UCR ECG example in its raw form.

It is to be noted that significant parts of a beat that are important to the present invention are the PR-interval, the QRS complex, the R-wave amplitude, and the ST interval.

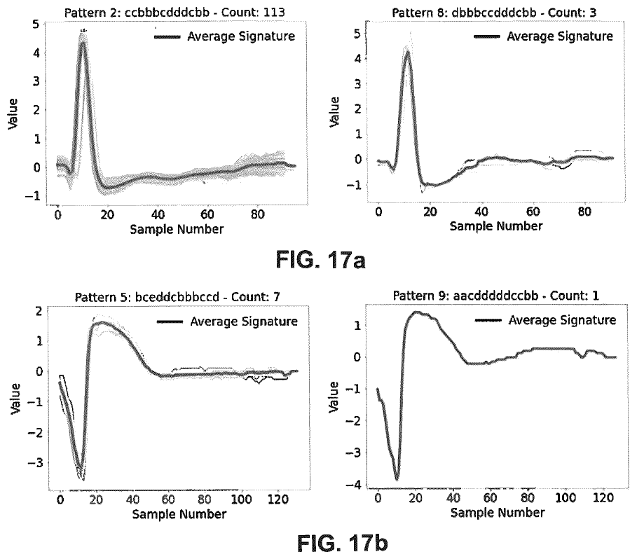

FIG. 17(a) shows two sets of normal pattern prototypes identified in the UCR ECG data. As shown herein, these patterns have captured 113 and 3 of the normal signals, respectively, while other normal signals have exhibited varying signatures and are consequently clustered under other patterns.

FIG. 17(b) depicts the PVC signals that are captured and clustered together. It is important to note one major difference in a T-wave portion (or the tail part) of Pattern 5 versus Pattern 9 that has likely resulted in their separate clustering. As noted in Pattern 5's average signature shows a compensatory pause, while Pattern 9 shows a parabolic trend, thus creating a different signature.

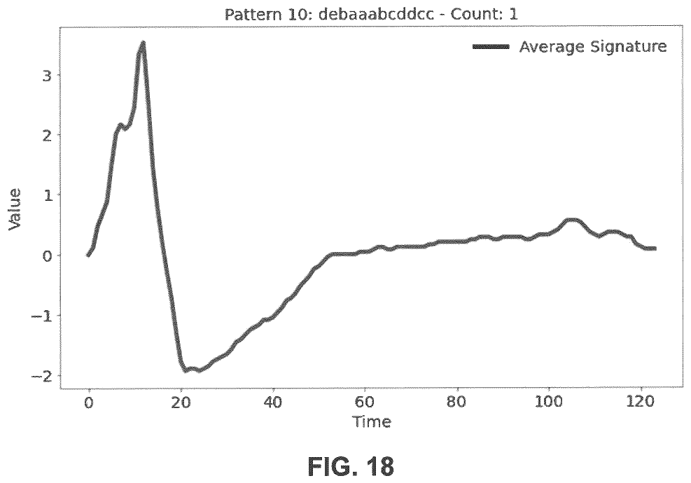

FIG. 18 depicts the anomalous signal captured by the framework of the present invention as Pattern 7. As noted in FIG. 18, the anomaly does not resemble the normal or the PVC prototypes and has a signature that is not similar to any other prototypes. This means that this pattern is identified as a separate prototype, thus forming a cluster of its own. While the UCR ECG example used herein only has one anomalous signal, it is very likely that if there were more anomalous signals, they would have been clustered with this pattern.

### 3.5 MIT-BIH Arrhythmia Dataset

The MIT-BIH Arrhythmia database is especially important since it contains real-world data and, more so, it includes labels that can be used to further verify the clustering results.

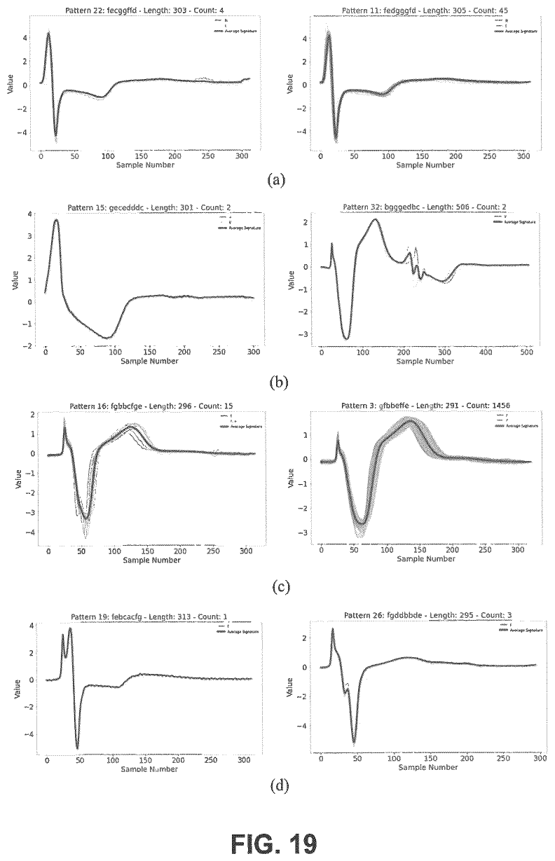

It is important to note that the f symbol is used by this dataset as a fusion or transition from one heartbeat rhythm to another. For example, this symbol can denote heartbeats that resemble Normal signals and those that resemble Paced beats. Additionally, the + symbol is used to identify the beginning of a trend and is usually preceded by another symbol such as f, /, V or N. As such, we can expect to see the for +symbols to appear in clusters of their own, as well as clusters formed by other prototypes, such as N or / symbols. Examples of this behavior are illustrated in FIG. 19, where a cluster of transitional (f) signals are clustered with the + signal, or the Nand f prototypes show similar behavior.

FIG. 19 depicts a special case in this analysis, where unusual patterns are singled out. As an example, pattern 19 on the left in FIG. 19 is uniquely identified as a single occurrence with an unusual PR interval and QRS complex that sets it apart from the others. Similarly, pattern 26 on the right in FIG. 19 has been identified to contain three f patterns, all of which are also unique in their behavior and unlike the other patterns. The clustering of similar patterns indicates that this framework is capable of accurately clustering similar patterns together while singling out the odd ones.

The right side of FIG. 19(c) shows a cluster of 1,456 paced pulses (symbol /) that are identified under the signature of gfbbeffe. A close inspection of these paced samples shows subtle differences between them and the transitional pulses (f) that are presented on the left side of FIG. 19(c), under pattern 16. Most notably, while both average curves show the QRS complex is observed around sample number 145, with a recorded R-wave amplitude of 1.5, the average behavior in the PR interval of the signal (around sample point 25) has an average amplitude less than 1 in the paced beats (FIG. 19(c), left plot), while the same interval shows an amplitude greater than 1 in fusion signals (FIG. 19(c), right plot).

Additionally, a visual inspection of the normal patterns (FIG. 19(a)) versus the PVC patterns (FIG. 19(b)) or the paced patterns (FIG. 19(c)) shows obvious differences between them. This indicates that the clustering module of the present invention is correctly identifying each pattern and clusters them under their respective prototypes.

FIG. 19(b) also shows that the framework of the present invention has been able to cluster the four PVC patterns correctly. It should be noted that two of the PVC patterns (shown in the left plot in FIG. 19(b)) have completely different behavior than the other two PVCs (right plot in FIG. 19(b)); yet the framework has picked up on these differences and has grouped them into their own clusters.

Since the MIT-BIH dataset is labeled, we are able to compare the clustered signals versus the ground-truth labels provided by the specialists. The accuracy of the framework in correctly identifying and clustering the signals was 2,185 out of 2,187 signals, or 99.90% in Sample 102 from the MIT-BIT dataset. It is also important to point out that the framework of the present invention has been able to correctly identify and cluster these signals in a self-supervised manner, for an example that is highly imbalanced, with 2,028 paced beats compared to only 99 normal beats, 4 PVCs and 56 fusion beats. This signifies that the framework of the present invention is not concerned about balanced data nor being trained on a specific type of signal (such as the normal ones or the PVCs).

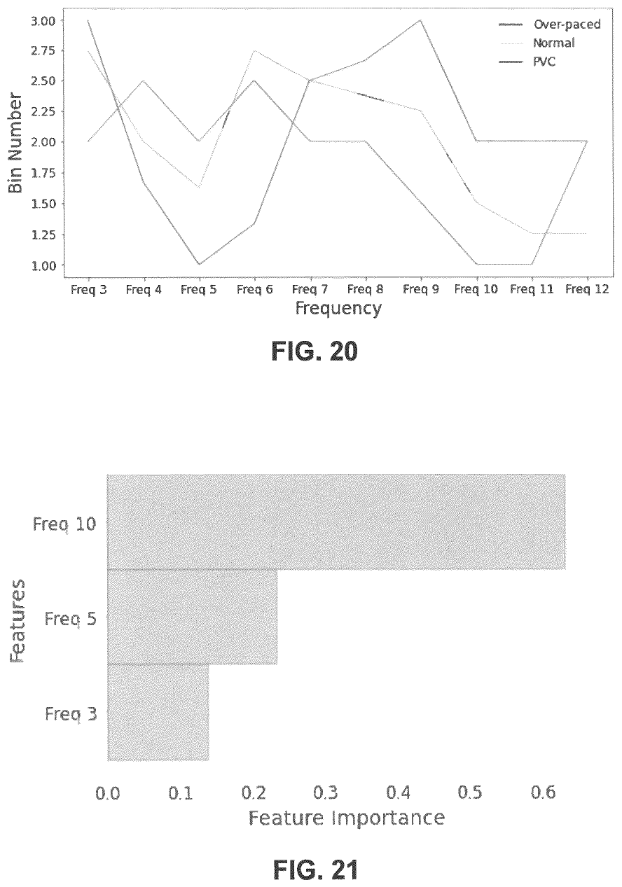

FIG. 20 depicts an overall look at the frequency bins that are formed by each pattern from MIT-BIH Sample 102. It can be observed that different prototypes exhibit significantly different frequencies; as such, it is expected that an ML algorithm trained on these prototypes can identify similarities or differences between them, and subsequently be used to extract rules that explain the underlying reasons for their behavior.

### 3.6 Rule Extractions

In order to explain the underlying differences between different prototypes and their signatures, a decision tree has been trained using the (FIGS) library on different prototypes that have been identified for the main categories in the MIT-BIH dataset. As noted hereinabove, the fusion signals (symbol f) and the start of signal indicators (symbol +) could be interpreted as any other trend, which would result in unintended errors being introduced to this model. For this reason, fusion and start indicators have been removed from the training set used for the decision tree. The FIGS. library has subsequently been used to extract the rules pertaining to this decision tree and visualized them accordingly.

Each prototype is composed of several different patterns, and each pattern covers a wide range of bin numbers. As a result, extracting a rule that would differentiate different prototypes is not a trivial task or something that can be done manually. FIG. 20 depicts the frequencies versus the average bin numbers that were observed for each prototype. While the average bin number may not be a good representative for each prototype, it shows an overall separation in features between different prototypes when it comes to their respective bin numbers that can be used to create decision rules. Once a decision tree is trained on these, the importance of different features in determining the leaves can be determined.

FIG. 21 depicts the top three most important features that are used in identifying different prototypes. As shown in FIG. 21, Frequency 10 plays the most pivotal role in differentiating paced beats from the normal or PVC ones, followed by Frequencies 5 and 3.

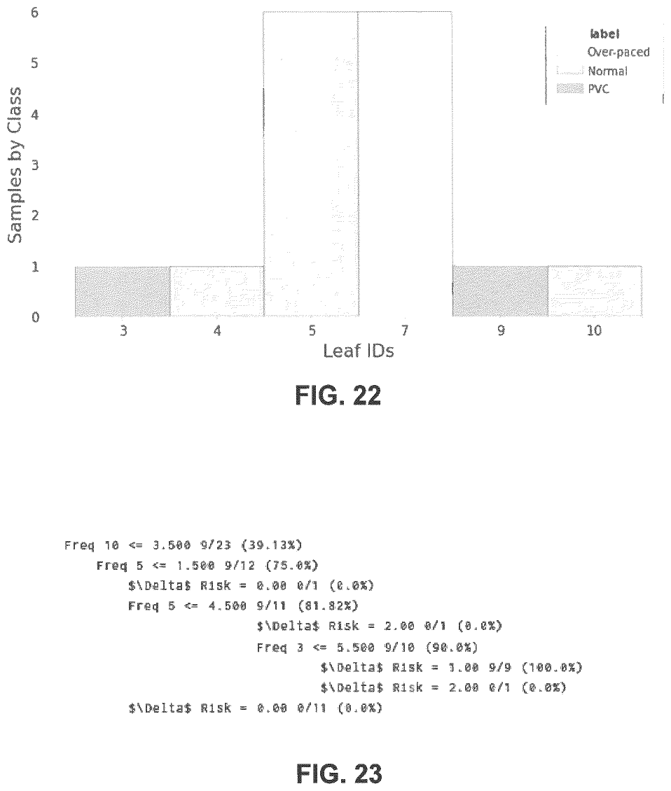

As discussed previously herein, the number of prototypes identified for each observed heartbeat type was two PVCs, nine normal and 12 paced prototypes. FIG. 22 depicts a breakdown of the number of unique prototypes versus their respective Leaf ID in the decision tree. It can be observed that the decision tree has been able to correctly identify each unique prototype in a separate leaf, as illustrated in FIG. 22.

FIG. 23 depicts a clear explanation of the decision rules that govern the decision tree created for the MIT-BIH sample 102. It is important to note that these sources of information should be used together to get a better understanding of the decision-making process followed by the model. The statistics and conditions presented in FIG. 23 help capture the decision-making process followed by our decision tree model. As illustrated in FIG. 23, the decision rule, expressed in terms of the most important features, indicates that a Freq. 10 bin number of over 3.5 will result in 11 out of the 12 observed paced prototypes, with no risk of guessing a wrong prototype (ΔRisk=0). If Freq. 10 bin number is less than 3.5, and Freq. 5 bin number is also less than 1.5, then the last remaining paced prototype is identified; otherwise, a Freq. 5 bin number greater than 4.5 (Freq. 10≤3.5) results in one of the PVC prototypes. Finally, if Freq. 10≤3.5 and 1≤Freq. 5≤4.5, and Freq. 3≤5.5, then we end up with all nine normal prototypes; otherwise, the last PVC prototype is determined.

### 4. Conclusion

Motifs, repeating sub-sequences in time series data, are crucial for summarizing and understanding system behavior over time. Existing motif detection algorithms, mostly supervised, struggle with variable-length motifs, noise sensitivity, and require pre-processing. The present invention introduces a self-supervised, scalable, and noise-resistant motif discovery framework applicable across various fields. The present invention also introduces the concept of “signature learning”, where instead of learning from raw data, the framework of the present invention will learn from motifs' signature. The present invention has demonstrated that this framework, which is based on signature extraction and a novel similarity metric, allows comparison of different length motifs and categorizes them into unique prototypes. This approach significantly reduces labeling costs by enabling domain experts to label unique motifs instead of each pattern, balancing data containing rare, normal, or abnormal patterns.

The framework of the present invention has been tested on ECG data with an exceptional accuracy of 99.90% in clustering and anomaly detection. This work also showcases that the method of the present invention explains motifs' characteristics using ensemble rule extraction and reveals insights into pattern behavior. This framework scales efficiently on both CPU and GPU platforms when handling large data sets. The versatility and high performance of the present invention mark a significant advancement in motif discovery, with wide applications in healthcare and predictive maintenance.

The above-described invention has a number of particular features that should preferably be employed in combination, although each is useful separately without departure from the scope of the invention. While the preferred embodiment of the present invention is shown and described herein, it will be understood that the invention may be embodied otherwise than herein specifically illustrated or described, and that certain changes in form and arrangement of parts and the specific manner of practicing the invention may be made within the underlying idea or principles of the invention.

What is claimed:

1. A technique capable of identifying an expected number of motifs in a given sequential or temporal set and extracting them, comprising:
(a) an algorithm that is used to identify a plurality of unique motifs and a signature of each motif; and
(b) a plurality—typically two (2)—main modules, comprising a Motif Extraction (ME) module and a Pattern Identification and Explanation (PIE) module.

2. The technique of claim 1, further comprising a distance metric that is used to compare a signature of each motif with a plurality of different levels of noise, thereby comparing a degree of resemblance between each motif.

3. The technique of claim 2, further comprising a self-supervised motif discovery to extract all meaningful motifs and categorize them together.

4. The technique of claim 3, wherein said technique provides advanced knowledge in motif analysis and incorporates additional explainability in order to highlight the differences between each motif signature.
